(19) United States
(12) Patent Application Publication
(10) Pub. No.: US 2006/0204476 A1
(43) Pub. Date: Sep. 14, 2006

(54) Treating hepatocellular carcinomas using therapeutic viruses

(76) Inventor: Michael S. Roberts, Myersville, MD (US)

Correspondence Address: LEWIS J. KREISLER, LEGAL DEPARTMENT, 930 CLOPPER ROAD, GAITHERSBURG, MD, 20878, US

(21) Appl. No.: 10/554,337

(22) PCT Filed: Apr. 14, 2004
(86) PCT No.: PCT/US04/11447

§ 371 Date: Oct. 24, 2005

Related U.S. Application Data
(60) Provisional application No. 60/465,622, filed on Apr. 25, 2003.

(51) Int. Cl. A61K 48/00

(52) U.S. Cl. 424/93.2

(57) ABSTRACT

An interferon-sensitive, replication-competent RNA virus is used to treat a mammalian subject having a hepatocellular carcinoma tumor.

### BACKGROUND OF THE INVENTION

The treatment of carcinomas with viruses is disclosed in WO 00/62735.

### SUMMARY OF THE INVENTION

This invention provides a method for treating a mammalian subject having a hepatocellular carcinoma tumor, comprising administering to the subject an amount of a therapeutic virus effective to treat the tumor, wherein the virus is an interferon-sensitive, replication-competent RNA virus.

This invention is based on the finding that an interferon-sensitive, replication-competent RNA virus, such as Newcastle disease virus, has effective anti-tumor activity against hepatocellular carcinoma cell types. Viral-mediated anti-neoplastic activity in vitro is correlated with effective anti-tumor activity in vivo. For example, Pecora et al. (1) show that the PV701 strain of Newcastle disease virus can cause complete regression of a squamous cell carcinoma and partial regression of colon cell carcinoma. Measurable tumor reduction in patients with mesothelioma, melanoma, breast carcinoma, pancreatic carcinoma, and neuroendocrine tumor was also observed (1). Each of these tumor types were shown to be sensitive to killing by Newcastle disease virus (PPMK107) by in vitro experimentation (WO 00/62735). Other viruses have also shown correlations between in vitro activity and clinical efficacy. For example, the G207 strain of herpes simplex virus showed oncolytic activity against glioblastoma tumor cells in vitro (2) and has demonstrated signs of anti-tumor activity against in patients with glioblstoma (3). Onyx-015 was shown to have in vitro activity against squamous cell carcinoma (4). In clinical testing against this same tumor type, patients treated with Onyx-015 experienced partial and complete tumor regressions (5). Similarly, the oncolytic virus CV706 showed activity against prostate tumors in vitro (6) and in clinical trials (7).

### BRIEF DESCRIPTION OF THE FIGURES

FIG. 1: TC₅₀ Value for HepG2 Cells Following Infection with Mesogenic Newcastle disease virus (PPMK107).

### DETAILED DESCRIPTION OF THE INVENTION

As used herein the transitional term “comprising” is open-ended. A claim utilizing this term can contain elements in addition to those recited in such claim. Thus, for example, the claims can read on treatment regimens that also include other therapeutic agents or therapeutic virus doses not specifically recited therein, as long as the recited elements or their equivalent are present.

As used herein “NDV” is an abbreviation for Newcastle disease virus. As used herein “DLT” is an abbreviation for dose limiting toxicity. As used herein the term “plaque-forming unit” (PFU) means one infectious virus particle. As used herein “BPFU” means billion PFUs. As used herein “PP” means plaque-purified. Thus, for example PPMK107 means plaque-purified Newcastle disease virus strain MK107. As used herein “PFU/m²”, which is a standard unit for expressing dosages, means PFUs per square meter of patient surface area. As used herein the term “replication-competent” virus refers to a virus that produces infectious progeny in cancer cells.

In accordance with the methods of this invention, when the therapeutic virus utilized is a Newcastle disease virus, the virus can be of low (lentogenic), moderate (mesogenic) or high (velogenic) virulence. The level of virulence is determined in accordance with the Mean Death Time in Eggs (MDT) test. (Alexander, “Chapter 27: Newcastle Disease” in Laboratory Manual for the Isolation and Identification of Avian Pathogens, 3^{rd} ed., Purchase, et al. eds. (Kendall/Hunt, Iowa), page 117.) Newcastle disease viruses are classified by the MDT test as lentogenic (MDT>90 hours); mesogenic (MDT from 60-90 hours); and velogenic (MDT<60 hours).

Any conventional interferon-sensitive, replication-competent RNA virus can be utilized in accordance with this invention to treat a mammalian subject having a hepatocellular carcinoma tumor. In an embodiment of the method of this invention, the interferon-sensitive, replication-competent RNA virus is negative-stranded. In progressively more specific embodiments, the negative-stranded RNA virus is a Paramyxovirus, for example a Newcastle disease virus, and more specifically a mesogenic strain of Newcastle disease virus.

In accordance with this invention, any conventional route or technique for administering viruses to a subject can be utilized. For example, the virus can be administered by the following routes: buccal, sublingual, enteral, rectal; and parenteral routes including subcutaneous, percutaneous, intradermal, intratumoral, peritumoral, intracranial, intrathecal, intramuscular, inhalation, intranasal, intrapleural, intrabronchial, endoscopic, vaginal, epidural, local, and topical; and systemic routes including intravenous, intraarterial, and intraperitoneal. When the virus is an enterovirus or a reovirus the oral route is also suitable. In one embodiment of this invention, the virus is administered systemically, preferably intravenously. For intravenous administration of a therapeutic virus in accordance with this invention, preferably the virus is a mesogenic strain of Newcastle disease virus.

It has been found that undesired side effects can be decreased by controlling the rate at which the virus is administered. When administering a mesogenic strain of Newcastle disease virus by the intravenous route, is preferable for a dose of the virus to be administered over an administration time period of up to 24 hours; and the dose to be administered at a rate of up to 7.0×10⁸ PFU per square meter of patient surface area in any ten minute sampling time period within the administration time period. More preferably, the rate at which the dose is administered is up to 2.0×10⁸ PFU per. square meter of patient surface area in any ten minute sampling time period within the administration time period. Generally it is convenient to select the rate of administration so that the administration time period is at least 1 hour. Still fewer side effects are generally observed when the administration time period is at least 3 hours.

In one embodiment of this invention, the therapeutic virus is administered to the subject in one or more cycles, wherein at least one cycle comprises administering sequentially one or more desensitization doses of the virus followed by administering one or more escalated doses of the virus, wherein the amount of the virus in each escalated dose is higher than the amount of virus in each desensitization dose. In a more specific embodiment, the cycle comprises one desensitization dose of at least 1.2×10¹⁰ PFU per square meter of patient surface area, and one or more escalated doses of at least 2.4×10¹⁰ PFU per square meter of patient surface area. A regimen utilizing desensitization and escalated doses can be combined with the technique described above of controlling the rate of administration of one or more of the doses. It is especially helpful to control the rate at which the first desensitization dose of the virus is administered.

In accordance with the method of this invention, optionally the therapeutic virus can be administered in combination with another anti-tumor agent such as those described in WO 00/62735 (page 36, line 20 to page 37, line 10).

The subject that is treated in accordance with this invention can be either a human subject or a non-human mammalian subject.

Although monitoring the treatment is not an essential aspect of the invention, there are techniques for measuring the therapeutic effects of the treatment. These include, measuring the size of the tumor after administration of the virus, and a decrease in tumor size is a positive result.

The invention will be better understood by reference to the following examples, which illustrate but do not limit the invention described herein. In the following examples the NDV used was a triple-plaque purified attenuated (mesogenic) version of the MK107 strain of Newcastle disease virus, described more fully in International Patent Publication WO 00/62735, published Oct. 26, 2000 (Pro-Virus, Inc.). The entire content of WO 00/62735 is hereby incorporated herein by reference.

### EXAMPLES

### Example 1

### Cytotoxicity Assay and Determination of TC₅₀

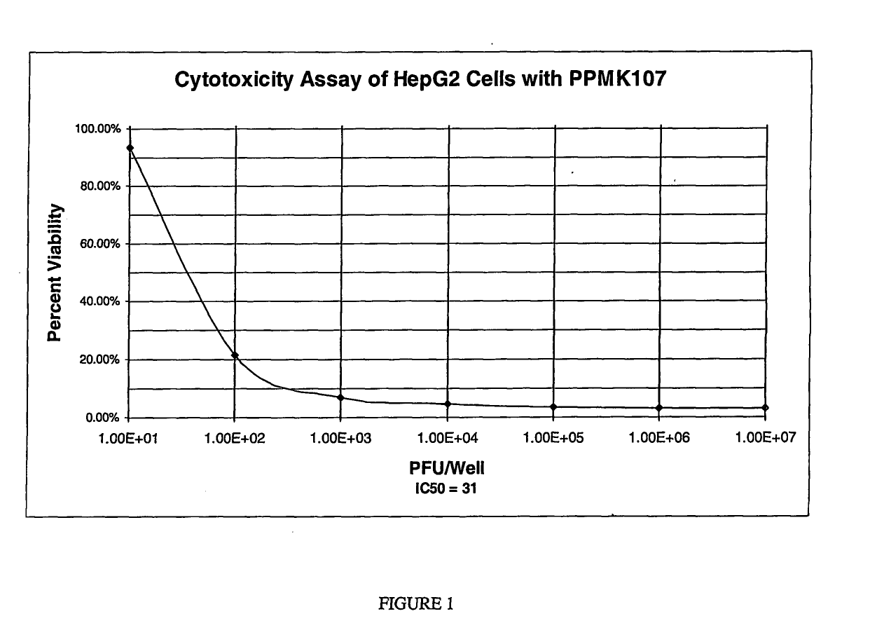

Hepatocellular carcinoma (HepG2) cells were assayed for sensitivity to killing by PPMK107. Cells were grown to approximately 70-80% confluence in 24-well tissue culture dishes. Growth medium was removed and PPMK107 was added to wells in 10-fold dilutions ranging from 1×10⁷ to 1 pfu/well. Control wells with no virus added were included on each plate. Virus was adsorbed for 90 minutes, virus was removed and replaced with medium, and then incubated for 5 days at 37° C. and 5% CO₂. Quantitative assessment of cell viability was performed using the CELLTITER 96 non-radioactive proliferation assay (PROMEGA). This assay is based on the conversion of the MT tetrazolium salt [3-[4,5-dimethylthiazol-2-yl]-2,5-diphenyl tetrazolium bromide] into a colored formazan product. The MTT dye solution was directly added to each well and incubated for 4 hours at 37° C. and 5% CO₂. Stop/solubilization solution (1 ml) was then added to each well. The plates were incubated overnight at 37° C. and 5% CO₂ and the absorbance at 570 nm was measured. The amount of signal is directly proportional to the number of viable cells in the well. The viability of the cells in the virus treated wells was expressed as a percent of the activity in the untreated control wells. The data were plotted graphically as PFU inoculated per well vs. cell viability as a percent of control. The TC₅₀ was calculated from the graphs determining the PFU/well resulting in 50% cell viability by minimizing the sum of squares after fitting the data to a four parameter logistic curve (FIG. 1).

### BIBLIOGRAPHY

1. Pecora A L, et al., J Clin Oncol 2002 May 1; 20(9):2251-66
2. Mineta T, et al., Nat Med 1995 September; 1(9):938-43
3. Markert J M, et al., Gene Ther 2000 May; 7(10):867-74
4. Heise C, et al., Nat Med 1997 June; 3(6):639-45
5. Nemunaitis J, et al., J Clin Oncol 2001 Jan. 15; 19(2):289-98
6. Chen Y, et al., Cancer Res. 2001 61: 5453-60.
7. DeWeese T L, et al., Cancer Res 2001 Oct. 15; 61(20):7464-72

What is claimed is:

1. A method for treating a mammalian subject having a hepatocellular carcinoma tumor, comprising administering to the subject an amount of a therapeutic virus effective to treat the condition, wherein the therapeutic virus is an interferon-sensitive, replication-competent, RNA virus.

2. The method of claim 1, wherein the virus is a negative-stranded RNA virus.

3. The method of claim 2, wherein the virus is a Paramyxovirus.

4. The method of claim 3, wherein the Paramyxovirus is a Newcastle disease virus.

5. The method of claim 4, wherein the virus is a mesogenic strain of Newcastle disease virus.

6. The method of claim 1, wherein the virus is administered systemically.

7. The method of claim 6, wherein the virus is administered intravenously.

8. The method of claim 7, wherein the virus administered is a mesogenic strain of Newcastle disease virus.

9. The method of claim 8, wherein the virus is administered over an administration time period of up to 24 hours; and the dose is administered at a rate of up to 7.0×10⁸ PFU per square meter of patient surface area in any ten minute sampling time period within the administration time period.

10. The method of claim 9, wherein the rate is up to 2.0×10⁸ PFU per square meter of patient surface area in any ten minute sampling time period within the administration time period.

11. The method of claim 9, wherein the administration time period is at least 1 hour.

12. The method of claim 11, wherein the administration time period is at least 3 hours.

13. The method of claim 8, wherein the therapeutic virus is administered to the subject in one or more cycles, wherein at least one cycle comprises administering sequentially one or more desensitization doses of the virus followed by administering one or more escalated doses of the virus, wherein the amount of the virus in each escalated dose is higher than the amount of virus in each desensitization dose.

14. The method of claim 1, wherein the cycle comprises one desensitization dose of at least 1.2×10¹⁰ PFU per square meter of patient surface area, and one or more escalated doses of at least 2.4×10¹⁰ PFU per square meter of patient surface area.

15. The method of claim 1, wherein the subject is a human subject.

16. The method of claim 1, wherein the subject is a non-human mammal.

17. The method of claim 1, wherein the size of the tumor decreases after administration of the virus.
